(19) United States
(12) Patent Application Publication
(10) Pub. No.: US 2010/0029612 A1
(43) Pub. Date: Feb. 4, 2010

(54) 2-Aminoethyl Substituted Pyrimidin-2-Ones Cyclopropanes, Pyrazolines, Pyrimidines and Benzothiazepines and Their Uses as Urotensin II and Somatostatin 5 Receptor Ligands

(76) Inventor: Roger Olsson, Bunkeflostrand (SE)

Correspondence Address: KNOBBE MARTENS OLSON & BEAR LLP, 2040 MAIN STREET, FOURTEENTH FLOOR, IRVINE, CA, 92614, US

(21) Appl. No.: 10/568,149

(22) PCT Filed: Feb. 18, 2004
(86) PCT No.: PCT/US04/04765

§ 371 Date: May 24, 2007

Related U.S. Application Data
(60) Provisional application No. 60/448,629, filed on Feb. 19, 2003.

(51) Int. Cl. A61K 31/554; A61K 31/513; A61K 31/416; A61K 31/16; C07C 233/51

(52) U.S. Cl. 514/211.09; 514/269; 514/407; 514/613; 540/546; 544/316; 548/379.4; 564/190

(57) ABSTRACT

The present invention provides a combinatorial approach to a library of novel compounds having four diversity points. The compounds provide for the mapping of urotensin II and somatostatin 5 receptors by differential binding of said receptors. The present invention further relates to a method of treating diseases for which modulation of the urotensin II receptor produces a physiologically beneficial response in said disease, such as those associated with CNS function and cardiovascular diseases. The present invention further relates to pharmaceutical compositions comprising these agents for the treatment of these diseases adapted to modulate the urotensin II receptor.

### FIELD OF THE INVENTION

The present invention provides a combinatorial approach to a library of novel compounds having four diversity points. The compounds provide for the mapping of urotensin II and somatostatin 5 receptors by differential binding of said receptors. The present invention further relates to a method of treating diseases for which modulation of the urotensin II receptor produces a physiologically beneficial response in said disease, such as those associated with CNS function and cardiovascular diseases. The present invention further relates to pharmaceutical compositions comprising these agents for the treatment of these diseases adapted to modulate the urotensin II receptor.

### BACKGROUND OF THE INVENTION

The design of drug-like chemical entities for non-biased screening constitutes an enormous challenge. Exploring the diversity represented by the amino acid side chains on nonpeptidic scaffolds has proven to be a powerful method for the design of ligands towards a wide range of targets. Recently, ligand-based drug design techniques were utilized for identification of novel nonpeptidic ligands at the somatostatin (SST) and urotensin II (UII) receptors.

A variety of disease states have been speculated to be associated with urotensin II and its receptor. However, the urotensin II peptide has yet to be directly associated to a disease state. Furthermore, disease states have yet to be directly linked to an altered function of the urotensin II receptor or the urotensin II peptide.

Human urotensin II has been reported as a potent spasmogen of primate airway smooth muscle and its contractile profile with pulmonary vascular tissue showed that there were regional differences in its efficacy, with potent contractile activity on pulmonary arteries whilst having no effect in tissues distal from the atria (Br. J. Pharmacol., 131(1); 10-12).

Human urotensin II (UII) has been reported as an endothelium-dependent vasodilator in rat small arteries (Br. J. Pharmacol.; 130(8); 1865-1870). The human urotensin II peptide acts as a vasoconstrictor of rat and primate aorta and induced a large increase in peripheral resistance in the circulation of primates along with a dramatic decrease in heart rate (Nature, 401; 282-286). In anesthetized rats urotensin II peptide induced a decrease in blood pressure (General and Comparative Endocrinology 64; 435-439, Neuroendocrinol. Lett. 14(5); 357-363). These results suggest that modulators of urotensin II and its receptor may alter cardiovascular function, particularly heart rate, cardiac output, peripheral resistance and arterial pressure.

Contemporaneously, Hacksell and co-workers published the first nonpeptide UII receptor agonist discovered by screening using the functional assay technology R-SAT (Croston G et al, J Med Chem 2002, 45, 4950).

It is notable that the discovered agonist resembles the minimalized UII peptide motif required for the biological activity, Tyr-D-Trp-Lys and Trp-Lys-Tyr, respectively. In addition to peptidomimetic design, the spatial arrangement of three amino acid side chains or analogs thereof has also been successful in proteomimetic design, mimicking the α-helix. Overall, these examples signify the importance of the subtle three-dimensional arrangement of the three amino acid side chains. This is especially evident in the case of somatostatin (SST) and UII ligands, where the same triad of pharmacophore elements results in activity at different receptors.

Combinatorial scaffold approaches have mainly been based on the decoration of core structures, e.g., dichloroheterocycles, or by formation of the skeleton during the addition of the diversity generating building blocks, i.e., diversity-oriented synthesis.

The work described herein provides a conceptually distinct methodology of combinatorial scaffolding built upon first generating the three necessary pharmacophore elements followed by constructing the central core unit as a fourth diversity point. This fourth diversity point is mainly the diverse spatial arrangement of the pharmacophore elements. The described methodology include the use of α,β-enones that previously have been used as branching points for the creation of drug-like heterocyclic libraries and therefore regarded as useful intermediates to set the stage for the construction of core structures (Marzinzik and Felder, J Org. Chem, 1998, 63, 723-727). However, a drawback is that most of the published synthetic procedures of α,β-enones only results in products with two diversity points. For example, α,β-enones has been used for the preparation of N-phenyl pyrazoline library (Powers et al, Tetrahedron 54, 4085-4096, 1998).

Recently, a practical and efficient multicomponent reaction was disclosed wherein substituted pyrrolidines and α,β-enones incorporating three diversity points could be synthesized (Bertozzi et al, Organic letters vol 4, 3147-3150, 2002, Bertozzi et al, Organic letters vol 4, 4333-4336, 2002). Advantageously, the α,β-enones with three diversity points can then be used as building block for the incorporation of a fourth diversity point.

Although, α,β-enones have been widely used for the creation of a range of heterocycles, only a few reported examples have incorporated α-substituents and to the best of our knowledge none with additional heteroatom functionalities such as basic amines. The synthesis of five new drug-like core structures (compounds of the general formula I to V) was selected to exemplify the use of α-substituted-α,β-enones as building block for providing compounds with agonistic activity towards somatostatin (SST) and urotensin II (UII) receptors.

### SUMMARY OF THE INVENTION

The work described herein provides data showing that a class of non-endogenous, non-peptide organic compounds such as α-substituted-α,β-enones of the general formula VI (compounds with three diversity points) and a number of compounds derivable from said α-substituted-α,β-enones such as those comprising an additional core of dihydropyrimidinone, pyrazoline or benzothiazepine possesses agonistic activity towards the human urotensin II receptor.

Quite remarkably, the class of compounds producing a biological response through the urotensin II receptor comprise four diversity points and have a core consisting of a dihydropyrimidinone, a cyclopropyl ketone, a pyrazoline, a pyrimidine or a benzothiazepine.

Accordingly, the invention relates in a first aspect to novel compounds of the general formula I to V or salts thereof,

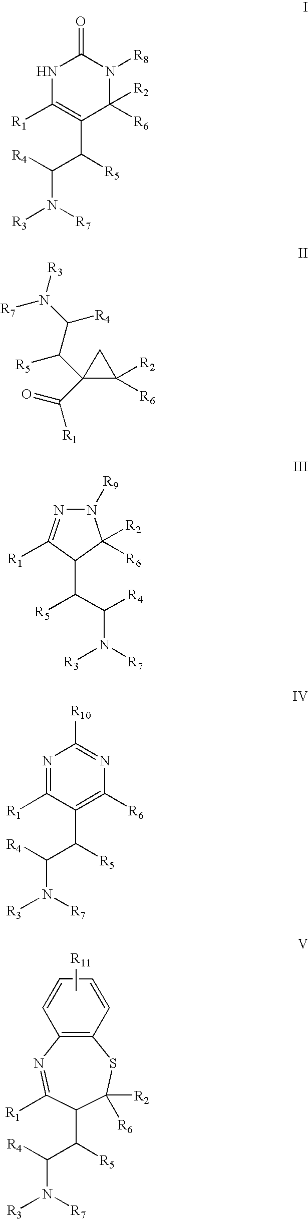

wherein R₁ and R₃ are independently selected from the group consisting of hydrogen, optionally substituted carbonyl(R), O(R), S(R), N(R)(R″), SO(R), SO₂(R), alkyl, alkenyl, alkynyl, cycloalkyl, heterocyclyl, aryl and heteroaryl, wherein these groups may be branched or unbranched and may be optionally substituted;

R₂ and R₄-R₆ are independently selected from the group consisting of hydrogen, optionally substituted O(R), S(R), N(R)(R″), alkyl, alkenyl, alkenyl, cycloalkyl, heterocyclyl, aryl and heteroaryl, wherein these groups may be branched or unbranched and may be optionally substituted;

R₇ is absent or selected from the group consisting of hydrogen, optionally substituted O(R), S(R), N(R)(R″), alkyl, alkenyl, alkynyl, cycloalkyl, heterocyclyl, aryl and heteroaryl, wherein these groups may be branched or unbranched and may be optionally substituted;

R₈ is selected from the group consisting of hydrogen, optionally substituted O(R), S(R), N(R)(R″), alkyl, alkenyl, alkynyl, cycloalkyl, heterocyclyl, aryl and heteroaryl, wherein these groups may be branched or unbranched and may be optionally substituted;

R and R″ are independently selected from the group consisting of hydrogen, optionally substituted alkyl, alkenyl or alkynyl, cycloalkyl, heterocyclyl, aryl and heteroaryl, wherein these groups may be branched or unbranched and may be optionally substituted;

R₉ and R₁₀ are selected from the group consisting of alkyl, alkenyl, alkynyl, cycloalkyl, heterocyclyl, aryl and heteroaryl, wherein these groups may be branched or unbranched and may be optionally substituted; and

R₁₁ is absent or selected from the group consisting of optionally substituted O(R), S(R), N(R)(R″), alkyl, alkenyl, alkynyl, cycloalkyl, heterocyclyl, aryl and heteroaryl, wherein these groups may be branched or unbranched and may be optionally substituted.

As stated, the above-mentioned compounds are provided with four diversity points and activate the UII and SST5 receptors. The work described herein further provides one- or two-step synthetic procedure for the achievement of such compounds with four diversity points using inexpensive and readily accessible starting materials.

Thus, in a further aspect, the invention relates to a method for the preparation of compounds of the general formula I to V, as defined herein, comprising the step of using a compound of formula VI,

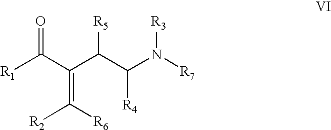

wherein R₁-R₇, R and R″ are as defined above.

Given that the compounds of formula I to V are agonists to the human urotensin II receptor and the somatostatin 5 receptor, a further aspect of the invention relates to a method for binding to the urotensin II receptor and/or somatostatin 5 receptor comprising the step of using one or more of the compounds of the general formula I to V, as defined herein.

Moreover, given that a variety of disease states have been speculated to be associated with urotensin II and its receptor, a further aspect of the invention relates to a method of treating diseases, and disorders for which activation or modulation of the urotensin II receptor produces a physiologically beneficial response in said disease or disorder comprising administering an effective amount of one or more of the compound(s) of formula I to V as defined herein to a mammal, such as a human. Within this scope, a still further aspect of the invention relates to compounds of the general formula I to V, as defined herein, for use as a medicament to a mammal including a human, such as a medicament for treating diseases and disorders for which activation or modulation of the urotensin II receptor produces a physiologically beneficial response in said disease or disorder.

Thus, in a further aspect the invention relates to a method of altering the vascular pressure in a mammal, comprising constricting or dilating vascular tissue in said mammal, the constricting or dilating is performed by the activation of urotensin receptor signalling, said activation being performed by the administration of an effective amount of one or more of compound(s) of the general formula I to V as defined herein to said mammal. Furthermore, a method of altering the heart rate in a mammal, comprising the activation of a urotensin receptor, said activating being performed by the administration of an effective amount of one or more of compound(s) of formula I to V, as defined herein, is anticipated. Finally, a method of altering the locomotor activity of a mammal, comprising administering to said mammal an effective amount of one or more of compound(s) of formula I to V, as defined herein, is an aspect of the invention.

A further aspect of the invention relates to a pharmaceutical composition comprising one or more of compound(s) of the general formula I to V as defined herein, together with pharmaceutically acceptable excipients and carriers.

### DETAILED DESCRIPTION OF THE PREFERRED EMBODIMENT

As stated, in a first aspect, the present invention relates to compounds of the general formula I to V or salts thereof (see the general formulas I to V above) derivable from the same intermediate product.

According to the invention R₁ and R₃ are independently selected from the group consisting of hydrogen, optionally substituted carbonyl(R), O(R), S(R), N(R)(R″), SO(R), SO₂(R), alkyl, alkenyl, alkynyl, cycloalkyl, heterocyclyl, aryl and heteroaryl, wherein these groups may be branched or unbranched and may be optionally substituted;

R₂ and R₄-R₆ are independently selected from the group consisting of hydrogen, optionally substituted O(R), S(R), N(R)(R″), alkyl, alkenyl, alkynyl, cycloalkyl, heterocyclyl, aryl and heteroaryl, wherein these groups may be branched or unbranched and may be optionally substituted;

R₇ is absent or selected from the group consisting of hydrogen, optionally substituted O(R), S(R), N(R)(R″), alkyl, alkenyl, alkynyl, cycloalkyl, heterocyclyl, aryl and heteroaryl, wherein these groups may be branched or unbranched and may be optionally substituted;

R₈ is selected from the group consisting of hydrogen, optionally substituted. O(R), S(R), N(R)(R″), alkyl, alkenyl, alkynyl, cycloalkyl, heterocyclyl, aryl and heteroaryl, wherein these groups may be branched or unbranched and may be optionally substituted;

R and R″ are independently selected from the group consisting of hydrogen, optionally substituted alkyl, alkenyl or alkynyl, cycloalkyl, heterocyclyl, alkyl and heteroaryl, wherein these groups may be branched or unbranched and may be optionally substituted;

R₉ and R₁₀ are selected from the group consisting of alkyl, alkenyl, alkynyl, cycloalkyl, heterocyclyl, aryl and heteroaryl, wherein these groups may be branched or unbranched and may be optionally substituted; and

R₁₁ is absent or selected from the group consisting of optionally substituted O(R), S(R), N(R)(R″), alkyl, alkenyl, alkynyl, cycloalkyl, heterocyclyl, aryl and heteroaryl, wherein these groups may be branched or unbranched and may be optionally substituted.

For the purpose of the current disclosure, the following definitions shall in their entireties be used to define technical terms, and shall also, in their entireties, be used to define the scope of the matter for which protection is sought in the claims.

The term “agonist” is defined as a compound that increases the activity of a receptor when it contacts the receptor.

The term “alkyl” is intended to mean a linear or branched saturated hydrocarbon chain, C₁₋₆-alkyl, wherein the longest chain has from one to six carbon atoms such as methyl, ethyl, n-propyl, isopropyl, n-butyl, isobutyl, sec-butyl, tert-butyl, pentyl, isopentyl, neopentyl, and hexyl.

The term “alkenyl” is intended to mean a linear or branched hydrocarbon group having from two to eight carbon atoms, C₂₋₈-alkenyl, and containing one or more double bonds. Illustrative examples of C₂-C₈-alkenyl groups include alkyl, homo-alkyl, vinyl, crotyl, butenyl, pentenyl, hexenyl, heptenyl and octenyl. Illustrative examples of C₂₋₈-alkenyl groups with more than one double bond include butadienyl, pentadienyl, hexadienyl, heptadienyl, heptatrienyl and octatrienyl groups as well as branched forms of these. The position of the unsaturation (the double bond) may be at any position along the carbon chain.

In the present context the term “alkynyl” is intended to mean a linear or branched hydrocarbon group, C₂₋₈-alkynyl, containing from two to eight carbon atoms and containing one or more triple bonds. Illustrative examples of C₂₋₈-alkynyl groups include ethynyl, propynyl, butynyl, pentynyl, hexynyl, heptynyl and octynyl groups as well as branched forms of these. The position of unsaturation (the triple bond) may be at any position along the carbon chain. More than one bond may be unsaturated such that the “C₂₋₈-alkynyl” is a di-yne or enedi-yne as is known to the person skilled in the art.

The term “cycloalkyl” is intended to cover three-, four-, five-, six-, seven-, and eight-membered rings, i.e., C₃₋₈-cycloalkyl, comprising carbon atoms only, whereas the term “heterocyclyl” is intended to mean three-, four-, five-, six- seven-, and eight-membered rings wherein carbon atoms together with from 1 to 3 heteroatoms constitute said ring. The heteroatoms of such heterocyclyl groups are independently selected from oxygen, sulphur, and nitrogen.

The term “heterocyclyl” groups may further contain one or more carbonyl or thiocarbonyl functionalities, so as to make the definition include oxo-systems and thio-systems such as lactams, lactones, cyclic imides, cyclic thioimides, cyclic carbamates, and the like.

C₃₋₈-cycloalkyl and heterocyclyl rings may optionally contain one or more unsaturated bonds situated in such a way, however, that an aromatic π-electron system does not arise.

Heterocyclyl rings may optionally also be fused to aryl rings, such that the definition includes bicyclic structures. Preferred such fused heterocyclyl groups share one bond with an optionally substituted benzene ring. Examples of benzo-fused heterocycyl groups include, but are not limited to, benzimidazolidinone, tetrahydroquinoline, and methylenedioxybenzene ring structures.

Illustrative examples of preferred “C₃₋₈-cycloalkyl” are the carbocycles cyclopropane, cyclobutane, cyclopentane, cyclopentene, cyclopentadiene, cyclohexane, cyclohexene, 1,3-cyclohexadiene, 1,4-cyclohexadiene, cycloheptane, cycloheptene, 1,2-cycloheptadiene, 1,3-cycloheptadiene, 1,4-cycloheptadiene and 1,3,5 cycloheptatriene.

Illustrative examples, without limitation, of “heterocyclyls” are the heterocycles tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, thiazoline, thiazolidine, and 1,3-oxathiolane. Binding to the heterocycle may be at the position of a heteroatom or via a carbon atom of the heterocycle, or, for benzo-fused derivatives, via a carbon of the benzenoid ring.

The term “aryl” is intended to mean a carbocyclic aromatic ring or ring system. Moreover, the term “aryl” includes fused ring systems wherein at least two aryl rings, or at least one aryl and at least one C₃₋₈-cycloalkyl share at least one chemical bond. Illustrative examples of “aryl” rings include optionally substituted phenyl, naphthalenyl, phenanthrenyl, anthracenyl, tetralinyl, fluorenyl, indenyl, and indanyl. A preferred aryl group is phenyl. The term “aryl” relates to aromatic, preferably benzenoid groups connected via one of the ring-forming carbon atoms, and optionally carrying one or more substituents selected from halogen, hydroxy, amino, cyano, nitro, alkylamido, acyl, C₁-C₆ alkoxy, C₁-C₆ alkyl, C₁-C₆ hydroxyalkyl, C₁-C₆ aminoalkyl, C₁-C₆ alkylamino, alkylsulfenyl, alkylsulfinyl, alkylsulfonyl, sulfamoyl, or trifluoromethyl. As stated, preferred aryl groups are phenyl, and, most suitably, substituted phenyl groups, carrying one or two, same or different, of the substituents listed above. The preferred pattern of substitution is para and/or meta. Representative examples of aryl groups include, but are not limited to, phenyl, 3-halophenyl, 4-halophenyl, 3-hydroxyphenyl, 4-hydroxyphenyl, 3-aminophenyl, 4-aminophenyl, 3-methylphenyl, 4-methylphenyl, 3-methoxyphenyl, 4-methoxyphenyl, 3-cyanophenyl, 4-cyanophenyl, dimethylphenyl, naphthyl, hydroxynaphthyl, hydroxymethylphenyl, trifluoromethylphenyl, and alkoxyphenyl.

The term “heteroaryl” is intended to mean a heterocyclic aromatic group where one or more carbon atoms in an aromatic ring have been replaced with one or more heteroatoms selected from the group comprising nitrogen, sulphur, phosphorous and oxygen.

Furthermore, in the present context, the term “heteroaryl” comprises fused ring systems wherein at least one aryl ring and at least one heteroaryl ring, at least two heteroaryl rings, at least one heteroaryl ring and at least one heterocyclyl ring, or at least one heteroaryl ring and at least one C₃₋₈-cycloalkyl ring share at least one chemical bond.

The term “heteroaryl” is understood to relate to aromatic, C₂₋₆ cyclic groups further containing one O or S atom or Lip to four N atoms, or a combination of one O or S atom with up to two N atoms, and their substituted as well as benzo- and pyrido-fused derivatives, preferably connected via one of the ring-forming carbon atoms. Heteroaryl groups may carry one or more substituents, selected from halogen, hydroxy, amino, cyano, nitro, alkylamido, acyl, C₁₋₆-alkoxy, C₁₋₆-alkyl, C₁₋₆-hydroxyalkyl, C₁₋₆-aminoalkyl, C₁₋₆-alkylamino, alkylsulfenyl, alkylsulfinyl, alkylsulfonyl, sulfamoyl, or trifluoromethyl. Preferred heteroaryl groups are five- and six-membered aromatic heterocyclic systems carrying 0, 1, or 2 substituents, which may be the same as or different from one another, selected from the list above. Representative examples of heteroaryl groups include, but are not limited to, unsubstituted and mono- or di-substituted derivatives of furan, benzofuran, thiophene, benzothiophene, pyrrole, pyridine, indole, oxazole, benzoxazole, pyrazole, indazole, and tetrazole, which are all preferred, as well as furazan, 1,2,3-oxadiazole, 1,2,3-thiadiazole, 1,2,4-thiadiazole, triazole, benzotriazole, quinoline, isoquinoline, pyridazine, pyrimidine, purine, pyrazine, pteridine, pyrrole, phenoxazole, oxazole, isoxazole, oxadiazole, benzopyrazole, indazole, quinolizine, cinnoline, phthalazine, quinazoline, and quinoxaline. The most preferred substituents are halo, hydroxy, cyano, O—C₁-C₆-alkyl, C₁-C₆-alkyl, hydroxy-C₁-C₅-alkyl, and amino-C₁-C₆-alkyl.

When used herein, the term “O—C₁-C₆-alkyl” is intended to mean C₁-C₆-alkyloxy, or alkoxy, such as methoxy, ethoxy, n-propoxy, isopropoxy, n-butoxy, isobutoxy, sec-butoxy, tert-butoxy, pentyloxy, isopentyloxy, neopentyloxy and hexyloxy

The term “halogen” includes fluorine, chlorine, bromine and iodine.

The term “salts” is intended to mean pharmaceutically acceptable acid addition salts obtainable by treating the base form of a functional group, such as an amine, with appropriate acids such as inorganic acids, for example hydrohalic acids, typically hydrochloric, hydrobromic, hydrofluoric, or hydroiodic acid, sulfuric acid, nitric acid, phosphoric acid and the like, or organic acids, for example acetic, propionic, hydroacetic, 2-hydroxypropanoic acid, 2-oxopropanoic acid, ethandioic, propanedioic, butanedioic, (Z)-2-butenedioic, (E)-butenedioic, 2-hydroxybutanedioic, 2,3-dihydroxybutanedioic, 2-hydroxy-1,2,3-propanetricarboxylic, methanesulfonic, ethanesulfonic, benzenesulfonic, 4-methylbenzenesulfonic acid, cyclohexanesulfamic, 2-hydroxybenzoic, 4-amino-2-hydroxybenzoic, and other acids known to the skilled practitioner.

The term “optionally substituted” is intended to mean any substituent that replaces an hydrogen and is selected from the group consisting of halogen, hydroxy, amino, cyano, nitro, alkylamido, C₁-C₆ acyl, C₁-C₆ alkoxy, C₁-C₆ alkyl. Furthermore, the term “optionally substituted” is meant to relate to hydrogen atoms replaced by heteroatom-containing fragments, connected through a heteroatom or a carbon atom.

The term “substituted phenyl” is intended to mean phenyl groups, carrying one or two, same or different, of the substituents selected from halogen, hydroxy, amino, cyano, nitro, alkylamido, C₁-C₆ acyl, C₁-C₆ alkoxy, C₁-C₆ alkyl, C₁-C₆ hydroxyalkyl, C₁-C₆ aminoalkyl, C₁-C₆ alkylamino, alkylsulfenyl, alkylsulfinyl, alkylsulfonyl, sulfamoyl, or trifluoromethyl. The preferred pattern of substitution is para and/or meta.

In one embodiment of the invention, R₁ is phenyl or a substituted phenyl. Further interesting combinations of embodiments include those, wherein R₂, R₄ and/or R₅ is hydrogen. In other embodiments of the invention, R₃ and R₇ denote an acyclic carbon group independently selected from the group consisting of alkyl and alkenyl, preferably ethyl.

Still further embodiments of the invention relate to the compounds of the general formula I to V, wherein R₆ is an optionally substituted phenyl group, preferably wherein the phenyl group is substituted with a halogen, such as when R₆ is 4-chlorophenyl.

Other combined or individual embodiments of the invention relate to wherein R₈ is methyl, R₉ is methyl, R₁₀ is phenyl or an optionally substituted phenyl and/or wherein Rx, is absent.

Furthermore, in some embodiments the compounds of the present invention may be in the form of isomeric mixtures and in other embodiments the compounds of the present invention may be in the form of one diastereoisomer form.

As stated, the disclosed work provides a one- or two-step synthetic procedure for the synthesis of compounds of the general formula I to V as defined herein using inexpensive and readily available starting materials and intermediate products. Advantageously, the compounds of the general formula I to V as defined herein are obtained by the addition of well known and commercially available reactants such as N-methyl urea, dimethyloxosulfonium methylide, methyl hydrazine, benzamidine and 2-aminothiophenol to α-substituted-α,β-enones. The α-substituted-α,β-enones used herein may be obtained by a simple three component synthesis including 4-halo-benzaldehyde and cyclopropyl-phenyl-ketone as building blocks and treatment with a metal-iodide.

An illustrative example of the synthetic procedures for obtaining the presently interesting compounds of the general formula I to V is disclosed in the following scheme:

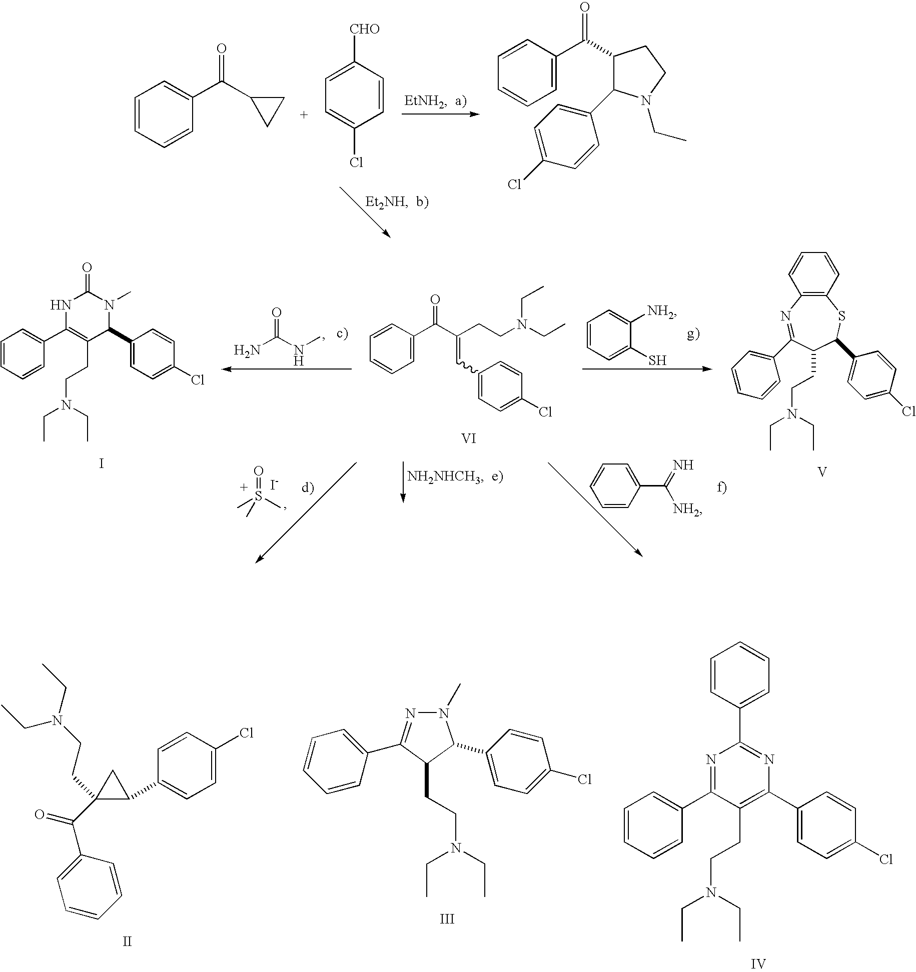

Thus, the disclosed invention relates in a second aspect to a method for the preparation of compounds of the general formula I to V, comprising the step of using a compound of formula VI,

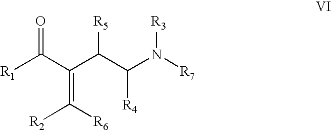

wherein R₁-R₇, R and R″ are as defined herein. The method further comprises the use of reactants selected from the group consisting of N-methyl urea, dimethyloxosulfonium methylide, methyl hydrazine, benzamidine and 2-aminothiophenol to obtain a compound of the general formula I, II, III, IV and V, respectively.

Given that the compound of formula VI may be obtained by a simple synthetic procedure as shown by the scheme shown above, a further aspect of the invention relates to a method for the preparation of compounds of the general formula I to V, comprising the step of using 4-halo-benzaldehyde and cyclopropyl-phenyl-ketone. Such a method may further include the use of a metal-iodide, such as a metal iodide is selected from the group consisting of Et₂Al-I or magnesium iodide.

Surprisingly, it was found that compounds of the general formula I to V are agonists to the human urotensin II receptor. Accordingly, a further aspect of the invention relates to a method for binding to the urotensin II receptor and/or somatostatin 5 receptor comprising the step of using one or more of the compounds of the general formula I to V as defined herein.

Moreover, given that a variety of disease states have been speculated to be associated with urotensin II and its receptor, a further aspect of the invention relates to a method of treating diseases and disorders for which activation or modulation of the urotensin II receptor produces a physiologically beneficial response in said disease or disorder comprising administering an effective amount of one or more of the compound(s) of the general formula I to V as defined herein to a mammal, such as a human.

Given the newly identified potential of compounds of the general formula I to V as defined herein, it is well within the scope of the invention to use a compound of the general formula I to V as defined herein for the preparation of a medicament for the treatment of diseases and disorders for which activation or modulation of the urotensin II receptor produces a physiologically beneficial response in a given disorder. Compounds of the present invention may be used for the preparation of a medicament to modulate the activity of proteins or pathways that produce beneficial physiological effects in many diseases. These may be diseases for which activation or modulation of the urotensin II receptor produces a physiologically beneficial response in said disease or disorder. The diseases may alternatively be associated with an imbalance of urotensin II and/or with an altered urotensin II receptor activity.

Such diseases may, at least in part, relate to diseases and disorders associated with CNS function, such as Parkinson's Disease, Alzheimer's Disease, amylotrophic lateral sclerosis, muscular dystrophy, childhood spinal muscular atrophy, progressive spinal muscular atrophy and progressive bulbar palsy; OPCA; ADHD; schizophrenia; sleep disorders such as insomnia, and autonomic dysfunctions such as Shy Drager syndrome.

Furthermore, diseases and disorders for which activation or modulation of the urotensin II receptor produces a physiologically beneficial response may relate to cardiovascular disorders such as hypertension; hypotensive states related to shock, sepsis, major surgery and/or congestive heart failure.

As stated, a variety of disease states have been suggested to be associated with either an altered functioning of the urotensin II receptor or to an imbalance of urotensin II. For example, alteration of urotensin II and signalling through its cognate receptor may be associated with, amongst other disease-states, both hypertension and hypotension. Accordingly, a further aspect of the invention relates to method of altering the vascular pressure in a mammal, comprising constricting or dilating vascular tissue in said mammal, said constricting or dilating being performed by the activation of urotensin receptor signaling, said activation being performed by the administration of an effective amount of one or more compounds the general formula I to V as defined herein. Similarly, the invention relates to methods of altering the heart rate in a mammal, comprising the modulation of urotensin receptor signaling, said modulation being performed by the administration of an effective amount of one or more compounds the general formula I to V as defined herein.

The surprising activity of the compounds of the general formula I to V renders them appropriate for use for the validation of the role of the urotensin II receptor as a drug target. Similarly, the invention relates to a method for augmenting cellular activity in a mammal, comprising activating the signaling of the urotensin II receptor, wherein the augmenting of said activity is performed by the administration to the mammal of a substance modulating the activity of said receptor, and the substance being administered in an amount effective to raise the concentration in the locality of the receptor of said substance to a level effecting a biological response through signaling of this receptor, the substance being a compound of the general formula I to V.

Moreover, the biological response induced by compounds of the general formula I to V, as defined supra, allow for the use of said compounds as agonist in antagonist assays with urotensin II receptor and/or somatostatin receptors. Furthermore, these biological responses produced as a result of the properties of compounds allows for the use of a compounds of the general formula I to V for the validation of the role of the urotensin II receptor as a drug target.

A further aspect of the invention relates to a pharmaceutical composition comprising one or more of compound of the general formula I to V, as defined herein, and pharmaceutically acceptable excipients or carriers formulated in a manner known to the skilled artisan such as according to formulations disclosed in Remington's Pharmaceutical Sciences. The composition may be formulated for oral administration, for administration via mucous membranes, or, amongst others parenteral administration in accordance with accepted practices.

The following examples teach the methods of the disclosed invention and the use of the resultant compounds. These examples are illustrative only and are not intended to limit the scope of the present invention. The treatment methods described below can be optimized using empirical techniques well known to those of ordinary skill in the art. Moreover, artisans of ordinary skill would be able to use the teachings described in the following examples to practice the full scope of the present invention.

### EXAMPLES

The invention is disclosed in further detail in the following non-limiting examples.

### Example 1

### Synthesis of Starting Material

### Compound of Formula VI

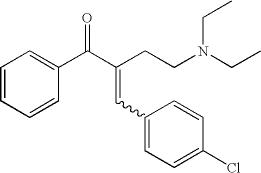

### General Procedure for the Et₂AlI-Promoted One-Pot Three-Component Synthesis of α-(Aminoethyl)-α,β-Enones, e.g., (E/Z)-2-(4-Chloro-benzylidene)-4-(2-diethylamino-ethyl)-1-phenyl-butan-1-one

In a 7 μl vial, at room temperature, 4-chloro-benzaldehyde (140 mg; 1.0 mmol; 1.0 eq.), Et₂Al-I (1.17 mL; 1.2 mmol; 1.2 eq.) and cyclopropyl-phenyl-ketone (146 mg; 138 μL; 1.0 mmol; 1.0 eq.) were added sequentially to a solution of diethylamine (73 mg; 104 μL; 1.0 mmol; 1.0 eq.) in CH₃CN (4.0 mL). The resulting mixture was vigorously shaken at room temperature overnight and then KOtBu (168 mg, 1.5 mmol, 1.5 eq.) was added. After 2 hours the reaction was quenched with saturated aqueous Na₂S₂O₃ solution (2 mL) and the mixture was extracted with EtOAc (5 mL). The organic phase was washed with saturated aqueous NaHCO₃ solution (2 mL) and brine (2 mL), dried over Na₂SO₄, filtered and concentrated. The corresponding crude reaction product was purified by flash chromatography on silica gel (CH₂Cl₂+MeOH 4%) to afford an 85:15 mixture of E/Z stereoisomers (204 mg; 60% yield) as an oil.

Data for the E/Z stereoisomeric mixture: R_{f}: 0.38 (silica gel, CH₂Cl₂+MeOH 5%); ¹H NMR (400 MHz, CDCl₃) δ 7.86-7.83 (m, 2H, Z); 7.81-7.77 (m, 2H, E); 7.58-7.53 (m, 1H, E); 7.49-7.43 (m, 3H); 7.40-7.34 (m, 4H, E); 7.31-7.29 (m, 2H, Z); 7.27-7.25 (m, 2H, Z); 7.06 (s, 1H, E); 7.05-7.02 (m, 2H, Z); 6.76 (s, 1H, Z); 2.96-2.89 (m, 2H); 2.74-2.68 (m, 6H); 2.63-2.50 (m, 8H); 1.01 (t, 6H, J=7.1 Hz, E); 0.97 (t, 6H, J=7.2 Hz, Z). ¹³C NMR (100 MHz, CDCl₃) δ 200.0 (Z); 198.8 (E); 141.3; 141.1; 139.9; 138.5; 136.1; 134.6; 134.5; 134.2; 133.4; 132.2; 130.6; 130.0; 129.9; 129.6; 129.0; 128.6; 128.5; 128.4; 51.9 (Z); 51.2 (E); 46.8 (E); 46.5 (Z); 34.7 (2C, Z); 25.7 (2C, E); 11.7 (2C, E); 11.5 (2C, Z). HRMS (Ion Mode: FAB⁺) Calcd. for C₂₁H₂₄ClNO (M⁺+1): 342.1624. Found: 342.1629. The diastereoselectivity was determined by integration of the peaks at δ 7.06 (isomer a) and δ 6.76 (isomer b).

### Example 2

### Reaction of Compound of Formula VI with N-methylurea Under the Formation of a Dihydropyrimidinone, 6-(4-Chloro-phenyl)-5-(2-diethylamino-ethyl)-1-methyl-4-phenyl-5,6-dihydro-3H-pyrimidin-2-one

### Compound of the General Formula I

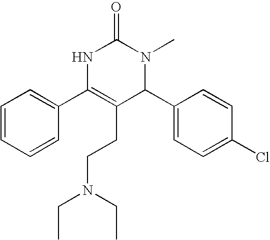

Reaction of N-methylurea with compound of formula VI of Example 1 at room temperature in the presence of NaOEt proceeded uneventfully and resulted in the dihydropyrimidinone as shown above in 48% yield as a single regioisomer. ¹H NMR experiments showed two singlets at 6.60 ppm and 4.48 ppm assigned as NH and H6, respectively, corroborating the previously assigned structure. The experimental conditions were as follows:

In a 20 mL vial, at room temperature, NaOEt (408 mg; 6.0 mmol; 6.0 eq) and N-methylurea (444 mg; 6.0 mmol; 6.0 eq.) were added sequentially to a solution of the compound of formula VI of Example 1 (341 mg; 1.0 mmol; 1.0 eq.) in DMF (10.0 mL) and the resulting mixture was vigorously shaken for 12 hours at room temperature. The reaction was then quenched with few drops of water and the mixture was washed with saturated aqueous NaHCO₃ solution (3 mL), brine (3 mL) and extracted with EtOAc (10 mL). The organic phase was dried over Na₂SO₄, filtered and concentrated. The corresponding crude reaction product was purified by flash chromatography on silica gel (CH₂Cl₂+MeOH 4% to 6%) to afford the substituted pyrimidine-2-one as shown above (193 mg; 48% yield) as an oil.

Data for: R_{f}: 0.41 (silica gel, CH₂Cl₂+MeOH 5%); ¹H NMR (400 MHz, CDCl₃) δ 7.41-7.24 (m, 9H); 6.60 (s, 1H, NH); 4.78 (s, 1H); 2.74 (s, 3H); 2.36 (ddd, 1H, J=15.8 Hz and 10.5 Hz and 5.2 Hz); 2.26 (q, 4H, J=7.2 Hz); 2.18 (ddd, 1H, J=15.5 Hz and 10.2 Hz and 5.5 Hz); 2.04 (ddd, 1H, J=15.9 Hz and 10.5 Hz and 5.3 Hz); 1.75 (ddd, 1H, J=15.4 Hz and 10.3 Hz and 5.2 Hz); 0.80 (t, 6H, J=7.3 Hz). ¹³C NMR (100 MHz, CDCl₃) δ 153.2; 140.1; 134.8; 134.3; 132.5; 129.3; 129.1; 129.0; 128.9; 128.7; 107.8; 66.3; 51.3; 46.8; 32.8; 25.9; 11.7. HRMS (Ion Mode: FAB⁺) Calcd for C₂₃H₂₈ClN₃O (M⁺+1): 398.2000. Found: 398.2004.

### Example 3

### Reaction of Compound of formula VI with Dimethyloxosulfonium Methylide Under the Formation of a Cyclopropyl Ketone, anti-1-Benzoyl-2-(4-chlorophenyl)-1-(2-diethylamino-ethyl)-cyclopropane

### Compound of the General Formula II

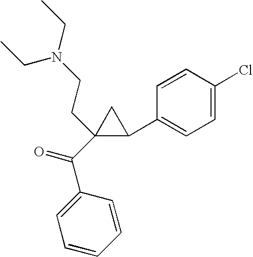

Reaction of excess dimethyloxosulfonium methylide with compound of formula VI of Example 1 resulted in the formation of cyclopropyl ketone 4 as the major product in 70% isolated yield. Only one diastereoisomer was indicated by NMR experiments, and the relative stereochemistry was determined to be anti by NOE measurements. Oxirane by-products were formed in minor amounts (<5%) according to LC/MS, probably due to the use of excess dimethyloxosulfonium methylide. When a stoichiometric amount of dimethyloxosulfonium methylide was used, a low conversion of the compound of formula VI was observed. The experimental conditions were as follows:

In a 20 mL vial, at room temperature, trimethylsulfoxonium iodide (616 mg, 2.8 mmol, 2.8 eq.) was added to a solution of NaH (110 mg, 2.4 mmol, 2.4 eq.; 55-60% in mineral oil) in DMSO (3.0 mL). The reaction mixture was flushed under a stream of argon and the vial was quickly capped. After 1 hour of shaking, the temperature was raised up to 60° C. and the vial was shaken for another hour. A solution of compound of formula VI (341 mg; 1.0 mmol; 1.0 eq.) in DMSO (2.0 mL) was then added drop wise to the suspension, and the mixture was kept at 60° C. After 3.5 hours the mixture was cooled to room temperature, quenched with water (20 mL) and extracted with EtOAc (3×25 mL). The collected organic phases were dried over Na₂SO₄, filtered and concentrated to give a crude reaction product (203 mg), which was purified by preparative HPLC to afford the major diastereoisomer as shown above in a >95:5 mixture (135 mg; 70% yield) as an oil.

Data; ¹H NMR (400 MHz, CDCl₃) δ 7.82-7.78 (m, 2H); 7.53-7.48 (m, 1H); 7.46-7.41 (m, 2H); 7.35-7.31 (m, 2H); 7.26-7.21 (m, 2H); 2.64 (dd, 1H, J=9.0 Hz and J=6.8 Hz); 2.32-2.24 (m, 1H); 2.21-2.12 (m, 5H); 1.92-1.86 (m, 1H); 1.85-1.78 (m, 1H); 1.51-1.42 (m, 1H) 1.32 (dd, 1H, J=6.8 Hz and J=5.1 Hz) 0.66 (t, 6H, J=7.1 Hz). ¹³C NMR (100 MHz, CDCl₃) δ 202.0; 137.4; 135.7; 132.8; 132.1; 130.3; 128.8; 128.6; 128.5; 50.4; 46.7; 37.0; 29.6; 28.5; 15.9; 11.0. HRMS (Ion Mode: FAB⁺) Calcd for C₂₂H₂₆ONCl (M⁺+1): 356.1781. Found: 356.1794.

### Stereochemical Assignment of Compound of Example 3 Via NOESY and NOE Spectroscopy

The anti/syn stereochemistry was determined by NOESY experiments on a pure major stereoisomer 4 (see figure below). The proton cis to H₂ (cis H₃) was determined through a NOESY experiment. Hereafter, it was possible to observe a NOE-correlation of H₂ with anti H_{3′}. Further NOE-correlations were observed between anti H_{3′}→H₄, anti H_{3′}→H₅ and anti H_{3′}→H₇.

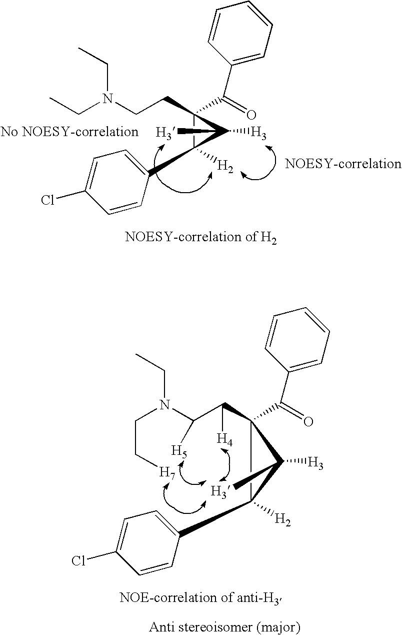

### Example 4

### Reaction of Compound of Formula VI with Methylhydrazine Under the Formation of a Pyrazoline, anti/syn-5-(4-Chloro-phenyl)-4-(2-diethylamino-ethyl)-1-methyl-3-phenyl-4,5-dihydro-1H-pyrazole

### Compound of the General Formula III

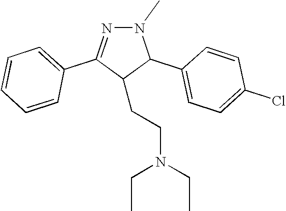

A pyrazoline scaffold was prepared by the condensation of compound of formula VI of Example 1 with methylhydrazine in the presence of L-InCl₃. This reaction resulted in 72% yield of the pyrazoline as shown above as a 3:1 diasteromeric mixture. The stereochemistry of the major isomer was confirmed as having an anti-configuration by the strong interaction between and the protons in the diethylamino chain and by the absence of any NOESY correlation between H4 (3.56 ppm) and H5 (3.98 ppm). Furthermore, the minor diastereoisomer had a strong NOESY correlation between H4 (3.58) ppm) and H5 (4.17 ppm), clearly indicating a syn configuration of this compound. Additionally, the pyrazoline core was stable to oxidation by air during storage. The experimental conditions were as follows:

In a 20 mL vial, at room temperature, methyl-hydrazine (268 μL; 230 mg; 5.0 mmol; 5.0 eq.) and InCl₃ (88 mg; 0.4 mmol; 0.4 eq.) were added to a solution of compound of formula VI of Example 1 (341 mg; 1.0 mmol; 1.0 eq.) in absolute EtOH (10.0 mL). The resulting mixture was vigorously shaken for 10 hours at 80° C. and then quenched with saturated aqueous NaHCO₃ solution (3 mL), extracted with EtOAc (15 mL) and washed with brine (3 mL). The organic phase was dried over Na₂SO₄, filtered and concentrated. The corresponding crude reaction product was purified by flash chromatography on silica gel (CH₂Cl₂+MeOH 4% to 6%) to give a 85:15 mixture of a anti/syn mixture of substituted dihydro-pyrazoles (264 mg; 72% yield) as an oil.

Data for the anti/syn mixture of dihydropyrazoles: R_{f}: 0.31 (silica gel, CH₂Cl₂+MeOH 5%); ¹H NMR (400 MHz, CDCl₃) δ 7.75-7.69 (m, 2H, syn); 7.59-7.56 (m, 2H, anti); 7.37-7.24 (m, 14H); 4.17 (d, 1H, J=9.4 Hz, syn); 3.98 (d, 1H, J=10.2 Hz, anti); 3.59-3.50 (m, 2H); 2.79 (s, 3H, syn); 2.78 (s, 3H, anti); 2.49-2.31 (m, 6H, anti); 2.28-2.21 (m, 2H, syn); 2.18-2.09 (m, 2H, syn); 2.01-1.86 (m, 2H); 1.81-1.72 (m, 2H); 1.61-1.52 (m, 1H, syn); 1.38-1.29 (m, 1H, syn); 0.87 (t, 6H, J=7.2 Hz, anti); 0.77 (t, 6H, J=7.2 Hz, sync). ¹³C NMR (100 MHz, CDCl₃) δ 155.4 (syn); 151.9 (anti); 139.8; 135.4; 133.8; 133.6; 133.1; 132.4; 129.8; 129.3; 129.1; 128.9; 128.8; 128.7; 128.5; 127.9; 126.6; 126.3; 77.5 (anti); 76.2 (syn); 54.1 (anti); 50.4 (syn); 50.1 (2C); 48.2 (syn); 46.9 (anti); 46.7 (syn); 41.6 (syn); 40.8 (anti); 28.5 (anti); 23.9 (syn); 11.7 (anti). HRMS (Ion Mode: FAB⁺) Calcd for C₁₂H₂₁ClN₃ (M⁺+1): 370.7050. Found: 369.2041.

### Stereochemical Assignment Via NOESY Spectroscopy

The anti/syn stereochemistry was determined by NOESY experiments on a 3:1 mixture of both stereoisomers a (major) and b (minor) (see figure below). In the major isomer (anti) strong NOESY correlations were observed between H₅→H₆ and H₅→H_{6′} furthermore NO NOESY correlations were observed between H₄→H₅. In the minor isomer (syn) strong NOESY correlations were observed between H₄→H₅, but NO NOESY correlations were observed between H₅→H₆ and H₅→H_{6′}.

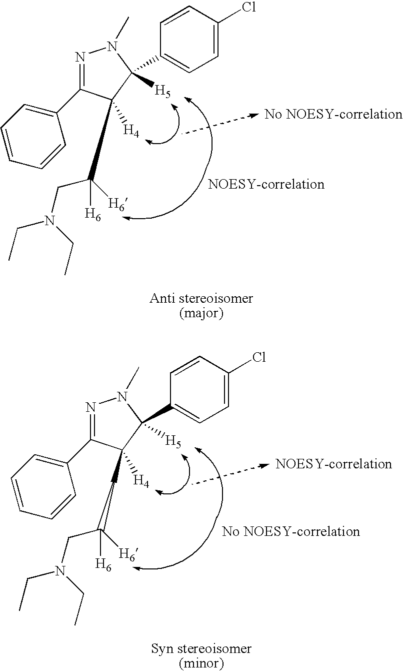

### Example 5

### Reaction of Compound of Formula VI with Benzamidine Under the Formation of a Pyrimidine, 4-(4-Chloro-phenyl)-5-(2-diethylamino-ethyl)-2,6-diphenyl-pyrimidine

### Compound of the General Formula IV

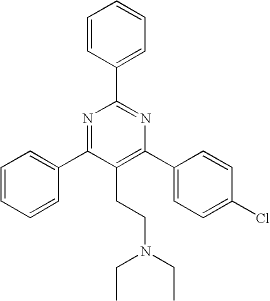

Treatment of compound of formula VI of Example 1 with benzamidine in DMF under an air atmosphere at 100° C. provided the pyrimidine as shown above in 53% yield. When the reaction was performed under an argon atmosphere, the corresponding non-aromatized dihydropyrimidine was obtained. Attempts to oxidize it further by vigorously stirring the reaction mixture at 100° C. under an air atmosphere were unsuccessful. Use of the corresponding HCl salt of benzamidine mainly resulted in a poor conversion, and the compound of formula VI was recovered. The experimental conditions were as follows:

In a 20 ml vial, at room temperature, benzamidine (720 mg; 6.0 mmol; 6.0 eq.) was added to a solution of compound of formula VI of Example 1 (341 mg; 1.0 mmol; 1.0 eq.) in DMF (10.0 mL). The resulting mixture was vigorously shaken for 12 hours at 100° C. under air atmosphere. The reaction was then quenched with few drops of water and the mixture was washed with saturated aqueous NaHCO₃ solution (3 mL), brine (3 mL) and extracted with EtOAc (10 mL). The organic phase was dried over Na₂SO₄, filtered and concentrated. The corresponding crude reaction product was purified by flash chromatography on silica gel (CH₂Cl₂+MeOH 3%) to afford the substituted pyrimidine as shown above (236 mg; 53% yield) as a solid.

Data: M.p.=90.5-92.3° C. (uncryst.); R_{f}: 0.33 (silica gel, CH₂Cl₂+MeOH 5%); ¹H NMR (400 MHz, CDCl₃) δ 8.53-8.45 (m, 2H); 7.66-7.59 (m, 4H); 7.53-7.48 (m, 5H); 7.46-7.42 (m, 3H); 2.98-2.92 (m, 2H); 2.25-2.18 (m, 2H); 2.14 (q, 4H, J=7.2 Hz); 0.59 (t, 6H, J=7.3 Hz). ¹³C NMR (100 MHz, CDCl₃) δ 168.2; 166.7; 161.7; 139.3; 138.1; 137.9; 137.7; 135.3; 130.6; 130.5; 129.2; 128.9; 128.8; 128.7; 128.6; 128.5; 51.5; 46.9; 25.2; 11.8. HRMS (Ion Mode: FAB⁺) Calcd for C₂₈H₂₈ClN₃ (M⁺+1): 442.2050. Found: 442.2046.

### Example 6

### Reaction of Compound of Formula VI with 2-aminothiophenol Under the Formation of a Benzothiazepine, anti-2-(4-Chloro-phenyl)-3-(2-diethylamino-ethyl)-4-phenyl-2,3-dihydro-benzo-[b]-[1,4]-thiazepine

### Compound of the General Formula V

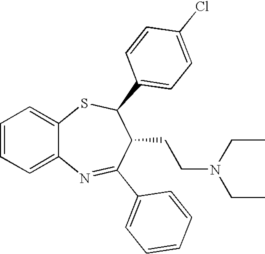

Reacting of compound of formula VI of Example 1 with 2-aminothiophenol in toluene in the presence of stoichiometric amount of p-toluenesulfonic acid resulted in a benzothiazepine scaffold. Other reaction conditions were tested, such as AcOH/MeOH or EtOH/reflux, PPh₃/acetone-water/rt, InCl₃/EtOH/reflux or Et₃N/EtOH/reflux were unsuccessful, resulting in either uncyclized Michael addition adduct or poor conversion. The lack of reactivity in the synthesis of this scaffold might be a reflection of the additional steric crowding in the trisubstituted enone. LC/MS analysis and NMR experiments indicated the formation of one diastereoisomer, which was determined to be anti by NOESY measurements. The detailed experimental conditions were as follows:

In a 20 ml vial, at room temperature, 2-aminothiophenol (534 μL; 625 mg; 5.0 mmol; 5.0 eq.) and p-toluenesulfonic acid monohydrate (190 mg; 1.0 mmol; 1.0 eq.) were added to a solution of compound of formula VI of Example 1 (341 mg; 1.0 mmol; 1.0 eq.) in toluene (10.0 mL) in the presence of 4 Å molecular sieves. The resulting mixture was refluxed for 24 hours and then quenched with saturated aqueous NaHCO₃ solution (3 mL), extracted with EtOAc (15 mL) and washed with brine (3 mL). The organic phase was dried over Na₂SO₄, filtered and concentrated. The corresponding crude reaction product was purified by flash chromatography on silica gel (CH₂Cl₂+MeOH 1% to 3%) to give one diastereoisomer of the substituted dihydro-benzothiazepine as shown above (201 mg; 45% yield) as an oil.

Data: R_{f}: 0.38 (silica gel, CH₂Cl₂+MeOH 5%); ¹H NMR (400 MHz, CDCl₃) δ 7.88-7.83 (m, 2H); 7.54-7.44 (m, 5H); 7.37-7.31 (m, 1H); 7.28-7.22 (m, 2H); 7.14-7.08 (m, 3H); 4.88 (d, 1H, J=11.5 Hz); 3.46-3.38 (m, 1H); 2.18-2.08 (m, 2H); 2.06-1.91 (m, 4H); 1.76-1.66 (m, 1H); 1.24-1.14 (m, 1H); 0.68 (t, 6H, J=7.2 Hz). ¹³C NMR (100 MHz, CDCl₃) δ 175.0; 152.1; 142.1; 139.3; 135.4; 133.7; 130.4; 130.0; 129.2; 128.7; 127.9; 127.8; 125.3; 124.7; 121.9; 65.4; 50.6; 47.6; 46.8; 28.3; 11.8. HRMS (Ion Mode: FAB⁺) Calcd for C₂₇H₂₉ClN₂S (M⁺+1): 449.1818. Found: 449.1819.

### Stereochemical Assignment Via NOESY Spectroscopy

The anti/syn stereochemistry was determined by NOESY experiments on the pure diastereoisomer (see figure below). Strong NOESY correlations were observed between H₂→H₄/H_{4′}; furthermore NO NOES) correlations were observed between H₂→H₃.

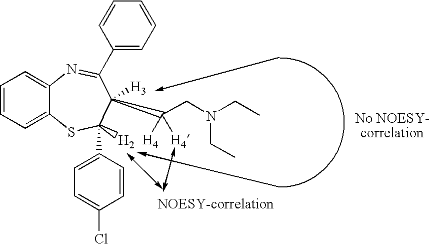

### Example 7

The compounds I to VI were tested as agonist at the UII and SST5 receptors in the functional mammalian cell-based assay R-SAT, described in U.S. Pat. Nos. 5,707,798, 5,912,132, and 5,955,281.

R-SAT assays were performed using NIH3T3 cells grown in tissue culture treated rollerbottles to 40-50% confluence. Cells were transfected for 12-16 hours with plasmid DNAs using SUPERFECT (QIAGEN) as per manufacture's protocols. R-SAT's were generally performed with 10 μg/rollerbottle of receptor and 50 μg/rollerbottle of beta-galactosidase plasmid DNA. All receptor and G-protein constructs used were in the PSI Mammalian Expression Vector (PROMEGA). The transfected cells were then trypsinized and frozen in DMEM containing 10% DMSO. Frozen cells were later thawed, plated at 10,000-40,000 cells per well of a 96½ area plate that contained drug. Cells were then grown in a humidified atmosphere with 5% ambient CO₂ for five days. Media was then removed from the plates and marker gene activity was measured by the addition of the beta-galactosidase substrate ONPG (in PBS with 5% NP-40). The resulting colorimetric reaction was measured in a spectrophotometric plate reader (Titertek Inc.) at 420 nM.

In these experiments, the starting material, compounds I, III and V were found to be partial to full agonists with similar potency as AC-7954 at the UII receptor. While the starting material and compound V displayed activity at both the UII and SST5 receptors, compounds I and III were selective UII agonists. The has synthesized illustrative examples of compound of the general formula I-V and found agonistic activity towards UII receptor.

TABLE 1
Agonist activity at the UII and SST5 receptors

| | |
|---|---|
| UII | SST5 |

| Compounds | Eff. | pEC₅₀ | Eff. | pEC₅₀ |
|---|---|---|---|---|
| AC-7954 | 120 | 5.7 | na | |
| Starting | 35 | 5.8 | 41 | 5.2 |
| material | | | | |
| I | 68 | 5.2 | na | |
| III | 31 | 5.4 | na | |
| V | 92 | 5.3 | 60 | 5.0 |

What is claimed is:

1. A compound of formula I, II, III, IV, V or salts thereof, 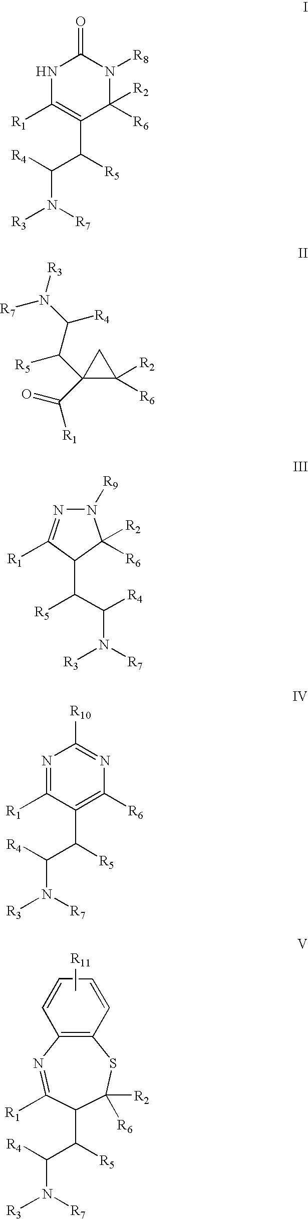
wherein R₁ and R₃ are independently selected from the group consisting of hydrogen, optionally substituted carbonyl(R), O(R), S(R), N(R)(R″), SO(R), SO₂(R), alkyl, alkenyl, alkynyl, cycloalkyl, heterocyclyl, aryl and heteroaryl, wherein these group may be branched or unbranched and may be optionally substituted;
R₂ and R₄, R₅ and R₆ are independently selected from the group consisting of hydrogen, optionally substituted O(R), S(R), N(R)(R″), alkyl, alkenyl, alkynyl, cycloalkyl, heterocyclyl, aryl and heteroaryl, wherein these groups may be branched or unbranched and may be optionally substituted;
R₇ is absent or selected from the group consisting of hydrogen, optionally substituted O(R), S(R), N(R)(R″), alkyl, alkenyl, alkynyl, cycloalkyl, heterocyclyl, aryl and heteroaryl, wherein these groups may be branched or unbranched and may be optionally substituted;
R₈ is selected from the group consisting of hydrogen, optionally substituted O(R), S(R), N(R)(R″), alkyl, alkenyl, alkynyl, cycloalkyl, heterocyclyl, aryl and heteroaryl, wherein these groups may be branched or unbranched and may be optionally substituted;
R₉ is selected from the group consisting of alkyl, alkenyl, alkynyl, cycloalkyl, heterocyclyl, aryl and heteroaryl;
R₁₀ is selected from the group consisting of alkyl, alkenyl, alkynyl, cycloalkyl, heterocyclyl, aryl and heteroaryl;
R₁₁ is absent or selected from the group consisting of optionally substituted O(R), S(R), N(R)(R″), alkyl, alkenyl, alkynyl, cycloalkyl, heterocyclyl, aryl and heteroaryl, wherein these groups may be branched or unbranched and may be optionally substituted; and
R and R″ are independently selected from the group consisting of hydrogen, optionally substituted alkyl, alkenyl or alkynyl, cycloalkyl, heterocyclyl, aryl and heteroaryl, wherein these groups may be branched or unbranched and may be optionally substituted.

2. (canceled)

3. (canceled)

4. (canceled)

5. (canceled)

6. The compound of claim 1, wherein R₁ is phenyl or a substituted phenyl.

7. The compound of claim 1, wherein R₂ is hydrogen.

8. The compound of claim 1, wherein R₄ and R₅ is hydrogen.

9. The compound of claim 1, wherein R₃ and R₇ is an acyclic carbon group independently selected from the group consisting of C₁-C₈ alkyl and C₁-C₈ alkenyl.

10. The compound of claim 9, wherein R₃ and R₇ is an ethyl group.

11. The compound of claim 1, wherein R₆ is an optionally substituted phenyl group.

12. The compound of claim 11, wherein R₆ is 4-chlorophenyl.

13. The compound of claim 1, wherein R₈ is methyl.

14. The compound of claim 1, wherein R₉ is methyl.

15. The compound of claim 1, wherein R₁₀ is phenyl or an optionally substituted phenyl.

16. The compound of claim 1, wherein R₁₁ is absent.

17. The compound of claim 1, wherein the compounds of formulae II, III and V are in the form of isomeric mixtures.

18. The compound of claim 1, wherein the compounds of formulae II, III and V are in the form of one diastereoisomer.

19. (canceled)

20. (canceled)

21. (canceled)

22. (canceled)

23. (canceled)

24. A pharmaceutical composition comprising a compound selected from the group consisting of 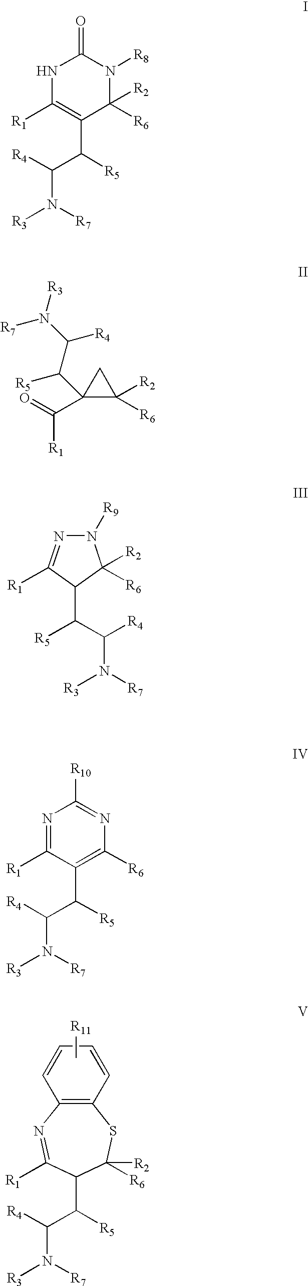
together with pharmaceutically acceptable excipients and carriers.

25. (canceled)

26. (canceled)

27. A method of treating diseases and disorders for which activation or modulation of the urotensin II receptor produces a physiologically beneficial response in said disease or disorder comprising administering an effective amount of a compound selected from the group consisting of 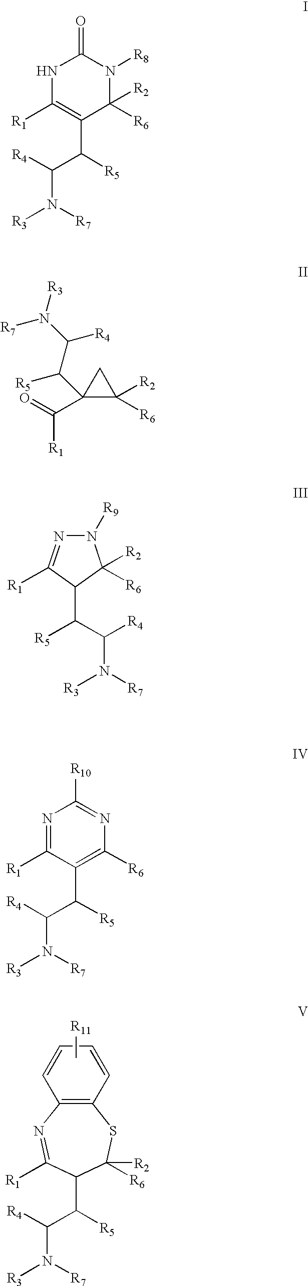
wherein the activation or modulation of the urotensin II receptor alters the vascular pressure, heart rate or locomotor activity in a mammal.

28. The method of claim 27, wherein the diseases and disorders are associated with CNS function, such as Parkinson's Disease, Alzheimer's Disease, amylotrophic lateral sclerosis, muscular dystrophy, childhood spinal muscular atrophy, progressive spinal muscular atrophy and progressive bulbar palsy; OPCA; ADHD; schizophrenia; sleep disorders such as insomnia, and autonomic dysfunctions such as Shy Drager syndrome.

29. The method of claim 27, wherein the diseases and disorders are cardiovascular disorders such as hypertension, hypotensive states related to shock, sepsis, major surgery and congestive heart failure.

30. (canceled)

31. (canceled)

32. (canceled)

33. (canceled)
